(19) United States
(12) Patent Application Publication
(10) Pub. No.: US 2022/0160911 A1
(43) Pub. Date: May 26, 2022

(54) ROTARY ULTRAVIOLET STERILIZING BOX

(71) Applicant: HERGY INTERNATIONAL CORP., Taipei City (TW)

(72) Inventors: Chun-Hung LU, Taipei City (TW); Chia-Te LIN, Taipei City (TW)

(21) Appl. No.: 16/953,392

(22) Filed: Nov. 20, 2020

(51) Int. Cl. A61L 2/10; A61L 2/24

(52) U.S. Cl. CPC A61L 2/10; A61L 2/24; A61L 2202/14; A61L 2202/122; A61L 2202/11

(57) ABSTRACT

This disclosure provides a rotary ultraviolet sterilizing box. A circuit board is disposed in the box. A rotary plate structure includes a motor and a carrier plate. The motor is disposed on a bottom of the box and electrically connected to the circuit board. The carrier plate is disposed in the box and driven by the motor to rotate. A linear LED light strip is arranged on an inner wall of the box and electrically connected to the circuit board. The linear LED light strip includes a plurality of ultraviolet LEDs arranged linearly and spacedly.

### BACKGROUND OF THE INVENTION

### Technical Field

The technical field relates to an ultraviolet sterilizing equipment, and more particularly relates to a rotary ultraviolet sterilizing box.

### Description of Related Art

Generally speaking, there may be a large number of bacteria in living environment or household items. Because these micro bacteria cannot be seen by eyes, they are easy to threaten human health. Moreover, as ultraviolet (UV) has an effect of destroying the nucleic acid, deoxyribonucleic acid (DNA) and ribonucleic acid (RNA) of microorganisms, it has been used in the field of eradicating bacteria and viruses for many years.

Furthermore, now there is an ultraviolet sterilizing box on the market, in which ultraviolet lamps (such as UV lamps or LEDs) are installed in a box to use ultraviolet light to irradiate objects in the box for a certain period of time to achieve the effect of sterilization. However, the ultraviolet lamp is fixed on one side of the box in the existing ultraviolet sterilizing box, thus the object in the box is only sterilized by ultraviolet radiation within a certain direction and range, so that the effect of sterilization cannot be fully achieved.

In view of the above drawbacks, the inventor proposes the present invention based on his expert knowledge and elaborate researches in order to solve the problems of prior art.

### SUMMARY OF THE INVENTION

It is an objective of this disclosure to provide a rotary ultraviolet sterilizing box, in which an ultraviolet irradiation is fully performed on the objects in the box to achieve a better effect of sterilization.

In order to achieve the object mentioned above, this disclosure provides a rotary ultraviolet sterilizing box including a box, a circuit board, a rotary plate structure and a linear LED light strip. The circuit board is disposed in the box. The rotary plate structure includes a motor and a carrier plate. The motor is disposed on a bottom of the box and electrically connected to the circuit board, and the carrier plate is disposed in the box and driven by the motor to rotate. The linear LED light strip is disposed on an inner wall of the box and electrically connected to the circuit board, and the linear LED light strip includes a plurality of ultraviolet LEDs arranged linearly and spacedly.

Comparing to the related art, the rotary ultraviolet sterilizing box of this disclosure includes a rotary plate structure and linear LED light strips. Therefore, the motor drives the carrier plate to rotate during operation to facilitate the linear LED light strips emitting ultraviolet light to irradiate the objects on the carrier plate. Moreover, the radiant energy of existing ultraviolet lamps or dotted ultraviolet LEDs is insufficient. The ultraviolet LEDs of the linear LED light strips in this disclosure are arranged linearly and spacedly, and include a primary and a secondary optical designs. Thereby, the radiant energy emitted by the ultraviolet LEDs may be concentrated to enhance illuminance, and the effect of sterilization is thus improved. Furthermore, the object may be fully and uniformly sterilized through the carrier plate rotating in 360 degrees to avoid blind spots for sterilization, and the practicality of this disclosure is improved.

### BRIEF DESCRIPTION OF DRAWINGS

The features of the invention believed to be novel are set forth with particularity in the appended claims. The invention itself, however, may be best understood by reference to the following detailed description of the invention, which describes a number of exemplary embodiments of the invention, taken in conjunction with the accompanying drawings, in which:

FIG. 1 is a perspective schematic view of the rotary ultraviolet sterilizing box of this disclosure.

FIG. 2 is a perspective exploded view of the rotary ultraviolet sterilizing box of this disclosure.

FIG. 3 is a front view of the rotary ultraviolet sterilizing box of this disclosure.

FIG. 4 is another embodiment of the rotary ultraviolet sterilizing box of this disclosure.

### DETAILED DESCRIPTION OF THE PREFERRED EMBODIMENTS

In cooperation with attached drawings, the technical contents and detailed description of the invention are described thereinafter according to a number of embodiments, being not used to limit its executing scope. Any equivalent variation and modification made according to appended claims is all covered by the claims claimed by the present invention.

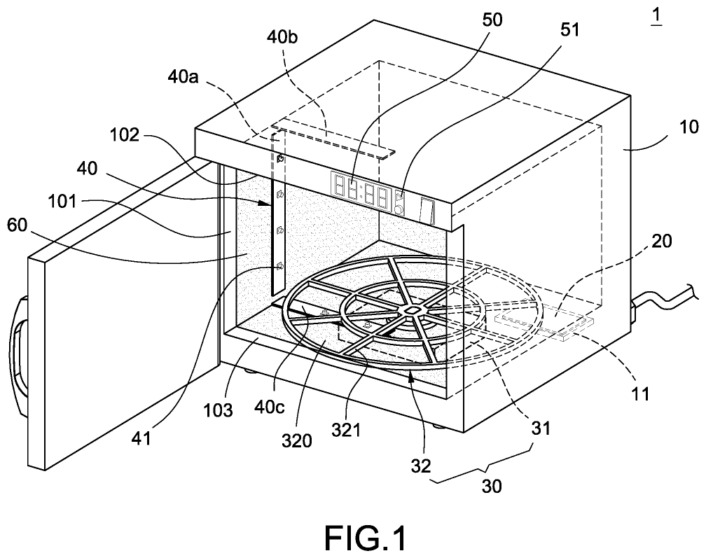

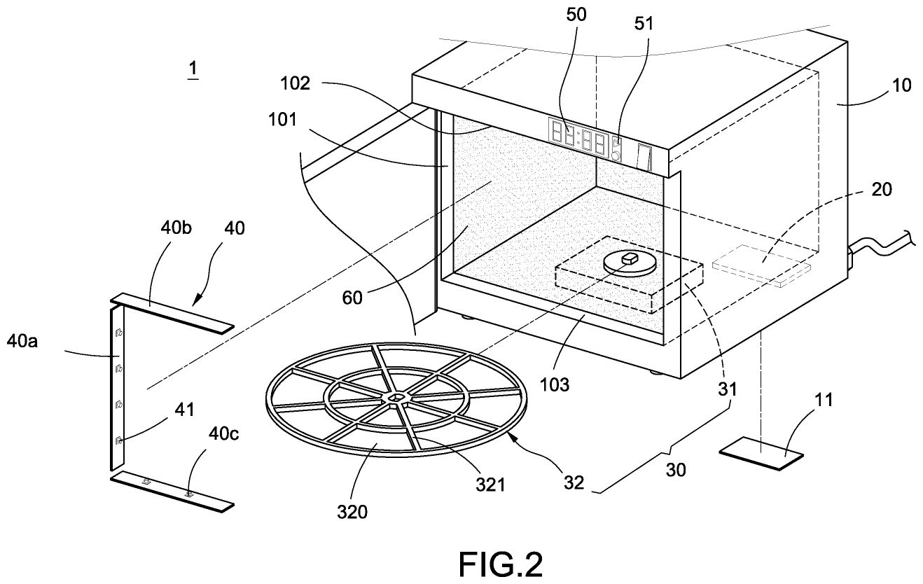

Please refer to FIG. 1 and FIG. 2, which depict a perspective schematic view and a perspective exploded view of the rotary ultraviolet sterilizing box of this disclosure. This disclosure provides a rotary ultraviolet sterilizing box including a box 10, a circuit board 20, a rotary plate structure 30 and a linear LED light strip 40. The circuit board 20, the rotary plate structure 30 and the linear LED light strip 40 are disposed in the box 10 respectively, and the circuit board 20 controls operations of the rotary plate structure 30 and the linear LED light strip 40. More detailed descriptions of the rotary UV sterilizing box 1 is as follows.

The box 10 is substantially rectangular, but this is not limited thereto. In one embodiment of this disclosure, the rotary ultraviolet sterilizing box 1 further includes a display panel 50 and an indicating light 51. The display panel 50 and the indicating light 51 are disposed on an outer surface of the box 10 and electrically connected to the circuit board 20.

It is worth noticing that, the display panel 50 is an LED digit display for displaying the local time or the operating time of the rotary ultraviolet sterilizing box 1. In addition, the indicating light 51 may be used as a warning light. When the ultraviolet illuminance emitted from the linear LED light strip 40 is insufficient, the indicating light 51 may be used as a reminder to notice user to pay attention to maintain the ability of sterilization of the linear LED light strip 40.

Specifically, the circuit board 20 is disposed on a bottom of the box 10. Preferably, the box further includes a lid 11. When the circuit board 20 is installed in the box 10, the lid 11 covers an outer side of the circuit board 20 so as to protect the circuit board 20 and maintain the safety during operation.

Moreover, the rotary plate structure 30 includes a motor 31 and a carrier plate 32. The motor 31 is disposed on the bottom of the box 10 and electrically connected to the circuit board 20. In addition, the carrier plate 32 is disposed in the box 10 and driven by the motor 31 to rotate. In the present embodiment, the carrier plate 32 is composed of a plurality of supporting ribs 321, and a plurality of hollow portions 320 are formed between the supporting ribs 321. In some embodiments, the carrier plate 32 may have a glass plate body.

The linear LED light strip 40 is disposed on one inner wall of the box 10 and electrically connected to the circuit board 20. The linear LED light strip 40 includes a plurality of ultraviolet LEDs 41 arranged linearly and spacedly (such as arranged in a straight line and disposed at intervals). It should be noted that the ultraviolet LEDs 41 include a primary and a secondary optical designs; thereby, the radiant energy emitted from the ultraviolet LEDs 41 is concentrated to enhance the illuminance and the effect of sterilization is improved.

Preferably, the number of the linear LED light strip is plural, and a plurality of linear LED light strips 40 are disposed on different inner walls of the box 10 respectively. The inner walls of the box 10 may at least include an inner side surface 101, an inner top surface 102 and an inner bottom surface 103.

Specifically, the linear LED light strips 40 include a first linear LED light strip 40a disposed on the inner side surface 101 of the box 10, a second linear LED light strip 40b disposed on the inner top surface 102 of the box 10 and a third linear LED strip 40c disposed on the inner bottom surface 103 of the box 10.

It is worth noticing that, the linear LED light strips 40 are arranged in an L-shape or a U-shape. In addition, the linear LED strips 40 are disconnected (or separated) from each other. It is not limited thereto, and the adjacent linear LED light strips 40 may be connected to each other.

It should be noted that, the rotary UV sterilizing box 1 of this disclosure may further include a reflective film 60. The reflective film 60 is attached to the inner wall, such as the inner side surface 101 or the inner top surface 102 etc., of the box 10. Thereby, the ultraviolet emitted from the linear LED light strips 40 is reflected by the reflective film 60 to improve the effect of sterilization.

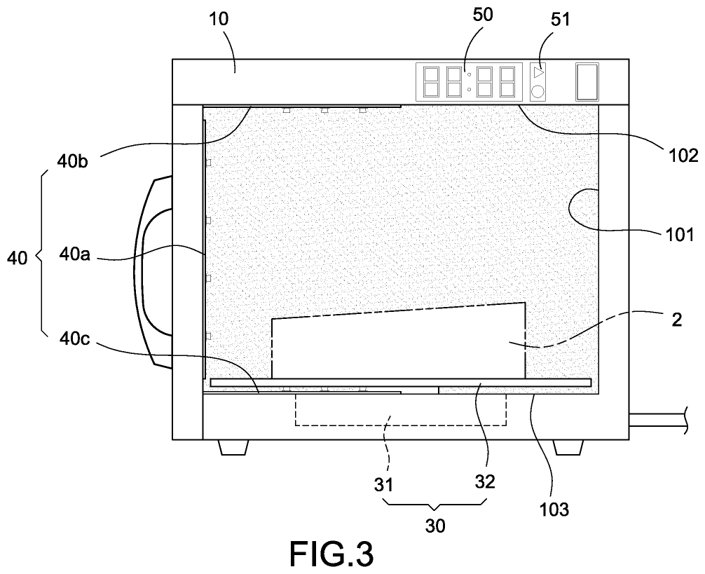

Please further refer to FIG. 3, it depicts a front view of the rotary UV sterilizing box of this disclosure. When the rotary ultraviolet sterilizing box 1 of this disclosure is being operated, an object 2 is placed on the carrier plate 32 for ultraviolet sterilization. When the operation is started, the circuit board 20 controls the rotary plate structure 30 and the linear LED light strip 40 to work.

When the operation is started, the motor 31 drives the carrier plate 32 to rotate. In addition, the linear LED light strip 40 emits ultraviolet to irradiate the object 2 on the carrier plate 32.

It should be noted that, the radiant energy of the linear LED light strip 40 of this disclosure is concentrated, so that it may enhance the illuminance and the effect of sterilization is improved. Furthermore, as the carrier plate 32 may be rotated in 360 degrees to avoid blind spots for sterilization; fully and uniformly irradiating may be achieved, and the effect of sterilization is improved.

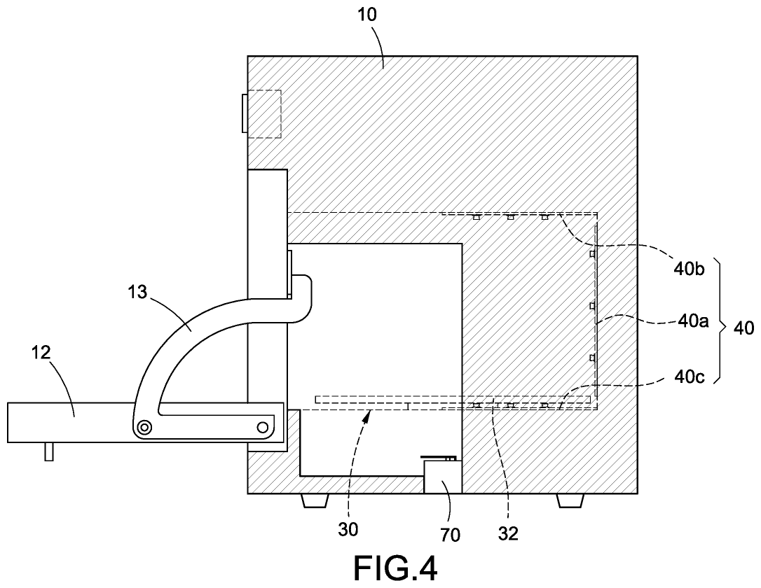

Please refer to FIG. 4, which depicts another embodiment of the rotary ultraviolet sterilizing box of this disclosure. In this embodiment, the door 12 of the box 10 is opened/closed in an upward/downward manner. In addition, the door 12 is connected with a supporting frame 13. On the other hand, a micro switch 70 is disposed in the box 10, and the micro switch 70 is electrically connected to the circuit board 20. Accordingly, when the door 12 is closed, one end of the supporting frame 13 abuts the micro switch 70. The micro switch 70 transmits a signal to the circuit board 20. Thus, the rotary plate structure 30 and the linear LED light strip 40 are activated, so as to enhance the safety in use.

Although the present invention has been described with reference to the preferred embodiment thereof, it will be understood that the invention is not limited to the details thereof. Various substitutions and improvements have been suggested in the foregoing description, and others will occur to those of ordinary skill in the art. Therefore, all such substitutions and improvements are intended to be embraced within the scope of the invention as defined in the appended claims.

What is claimed is:

1. A rotary ultraviolet sterilizing box, comprising:
a box;
a circuit board, disposed in the box;
a rotary plate structure, comprising a motor and a carrier plate, wherein the motor is disposed on a bottom of the box and electrically connected to the circuit board, and the carrier plate is disposed in the box and driven by the motor to rotate; and
a linear LED light strip, disposed on one inner wall of the box and electrically connected to the circuit board, wherein the linear LED light strip comprises a plurality of ultraviolet LEDs arranged linearly and spacedly.

2. The rotary ultraviolet sterilizing box according to claim 1, further comprising: a display panel and an indicating light, wherein the display panel and the indicating light are disposed on an outer surface of the box and electrically connected to the circuit board.

3. The rotary ultraviolet sterilizing box according to claim 1, wherein the circuit board is disposed on the bottom of the box, and the box further comprises a lid covering an outer side of the circuit board.

4. The rotary ultraviolet sterilizing box according to claim 1, wherein the carrier plate comprises a plurality of supporting ribs, and a plurality of hollow portions are disposed between the supporting ribs.

5. The rotary ultraviolet sterilizing box according to claim 1, further comprising: a reflective film, attached to the inner wall of the box.

6. The rotary ultraviolet sterilizing box according to claim 1, wherein the number of the linear LED light strip is plural, and a plurality of linear LED light strips are disposed on different inner walls respectively; and the linear LED light strips comprise a first linear LED light strip disposed on an inner side surface of the box, a second linear LED light strip disposed on an inner top surface of the box and a third linear LED strip disposed on an inner bottom surface of the box.

7. The rotary ultraviolet sterilizing box according to claim 6, wherein the linear LED light strips are arranged in an L-shape or a U-shape.

8. The rotary ultraviolet sterilizing box according to claim 1, wherein the box comprises a micro switch disposed therein, and the micro switch is electrically connected to the circuit board.

9. The rotary ultraviolet sterilizing box according to claim 6, wherein the linear LED strips are separated and disconnected from each other.

10. The rotary ultraviolet sterilizing box according to claim 1, wherein the ultraviolet LEDs comprise a primary optical design and a secondary optical design.
